(19) United States
(12) Patent Application Publication
(10) Pub. No.: US 2023/0189869 A1
(43) Pub. Date: Jun. 22, 2023

(54) HASHISH HAVING SUBSTANTIALLY UNIFORM CHARACTERISTICS AND METHOD FOR PRODUCING SAME AT AN INDUSTRIAL SCALE

(71) Applicant: HEXO OPERATIONS INC., Gatineau (CA)

(72) Inventors: François Chouinard, Blainville (CA); Renato Devien Durbano, Ottawa (CA); Jamie Savard, Gatineau (CA)

(21) Appl. No.: 17/925,591

(22) PCT Filed: May 17, 2021
(86) PCT No.: PCT/CA2021/050673

§ 371 Date: Nov. 15, 2022

Related U.S. Application Data
(60) Provisional application No. 63/025,863, filed on May 15, 2020.
(60) Provisional application No. 63/146,517, filed on Feb. 5, 2021.
(60) Provisional application No. 63/183,385, filed on May 3, 2021.
(60) Provisional application No. 63/185,210, filed on May 6, 2021.

(51) Int. Cl. A24B 15/16; A24B 15/30

(52) U.S. Cl. CPC A24B 15/16; A24B 15/303

(57) ABSTRACT

Traditional methods of hash production are very labor intensive, and the skills of the individual play a key role in defining the quality of the finished product. Hash production requires multiple steps and lacks automation, and thus, it is not easily scaled and very costly for commercial production. The present disclosure relates to hashish product comprising a cohesive mass of isolated cannabis trichomes and having substantially uniform characteristics and to a process of making same. The process comprises providing isolated cannabis trichomes; mixing the isolated cannabis trichomes under conditions sufficient to obtain a substantially homogeneous resinous mixture; and retrieving at least a portion of the mixture through an extrusion die to obtain the hash product.

### TECHNICAL FIELD

This application generally relates to the field of hashish products and methods of manufacturing hashish products at an industrial scale.

### BACKGROUND

With stage-wise legalization of cannabis-based consumer products in Canada and eventually in various other areas in the world, advancements in extraction technology, industrial scale production and accessibility to a wide variety of forms have accelerated to fulfill emerging demands.

Hashish (or hash) is a concentrated derivative of cannabis plants, the dried resin glands of the flowering tops of mature and unpollinated female cannabis plants. The resin glands are known as trichomes. It contains the same active ingredients as marijuana - including tetrahydrocannabinol (“THC”) and other cannabinoids - yet at higher concentration levels than the un-sifted buds or leaves from which dried marijuana is made, which is tantamount to higher potency. Hashish is typically used for recreational or medicinal (i.e., health and wellness) purposes.

The trichomes are substantially isolated from plant matter. The isolated trichomes are usually collected by hand, by mechanical beating of the plants or by submersing the cannabis plants in icy water and then using small sieves to isolate the trichomes. Alternatively, mechanical isolation may be used to isolate trichomes from plant matter, such as sieving through a screen by hand or in motorized tumblers, as described for example in WO 2019/161509. Isolated trichomes have a powder appearance (typically referred to as “kief”) and are pressed to obtain blocks of hashish, either by hand or with industrial presses.

The production of hashish is very labor intensive, and it is practically an art, where the skills of the individual play a key role in defining the quality and/or physical characteristics of the finished hashish product. Hashish production requires multiple steps and lacks automation, and thus, it is not easily scaled and very costly for commercial production. Since the cost of goods is a significant factor in securing and growing market share, particularly in the fight against the black market, the current methods for manufacturing hash at an industrial scale remain unsatisfactory.

Considering the above, it would be highly desirable to be provided with a hashish product and manufacturing method for making same that would at least partially alleviate the disadvantages of the existing technologies.

### SUMMARY

This Summary is provided to introduce a selection of concepts in a simplified form that are further described below in the Detailed Description. This Summary is not intended to identify key aspects or essential aspects of the claimed subject matter.

In one broad aspect, the present disclosure relates to a hashish product comprising a cohesive mass of isolated cannabis trichomes and having substantially uniform physical characteristics. For example, such uniform physical characteristics may characterize a hashish product unit per se provided herein. For example, the herein described uniform physical characteristics may be assessed within a single product unit and thus may characterize the hashish product unit per se. For example, such uniform physical characteristics may characterize the set of hashish product units provided herein per se. For example, the herein described uniform physical characteristics may be assessed within a set of a plurality of hashish product units and thus may characterize the set of hashish product units per se. For example, the herein described uniform physical characteristics may be assessed between a plurality of sets each set having a plurality of hashish product units and thus may characterize the plurality of sets per se.

In one broad aspect, the present disclosure relates to a set of hashish product units, wherein each hashish product unit in the set of hashish product units comprises a cohesive mass of isolated cannabis trichomes, and wherein a first hashish product unit in the set of hashish product units is characterized with a physical characteristic which is substantially identical to an average of the physical characteristic in the set of hashish product units.

For example, in some implementations, the set of hashish product units includes one or more of the following:
a) a respective cross section dimension which is substantially identical to a corresponding average cross section dimension in the set of hashish product units; b) a respective unit weight which is substantially identical to an average unit weight in the set of hashish product units; c) a respective density measured at 20° C. which is substantially identical to an average density measured at 20° C. in the set of hashish product units; or d) any combinations of a) to c).
the physical characteristic in about 85 percent or more, preferably about 90 percent or more, more preferably about 95 percent or more, of hashish product units in the set of hashish product units is substantially identical to the average of the physical characteristic in the set of hashish product units.
the physical characteristic being substantially identical represents from about 0.9 to about 1.1 times, preferably from about 0.95 to about 1.05 times, more preferably from about 0.98 to about 1.02 times, the average physical characteristic in the set of hashish product units.
each hashish product unit in the set of hashish product units comprises a cannabinoid content of from about 5 wt.% to about 90 wt.%, preferably from about 20 wt.% to about 90 wt.%.
the cannabinoid content comprises one or more of tetrahydrocannabinol (THC), cannabidiol (CBD), cannabigerol (CBG), cannabinol (CBN), and any combinations thereof.
the isolated cannabis trichomes are from one or more strain(s) of cannabis plant.
the first hashish product unit further comprises one or more additional component.
the one or more additional component comprises one or more cannabinoid(s), one or more terpene(s), one or more flavonoid(s), water, one or more flavoring agent(s), one or more non-toxic coloring agent(s), or any combinations thereof.
the one or more additional components is incorporated within the cohesive mass of the first hashish product unit.
the one or more additional components is distributed on at least a portion of a surface of the hashish product unit.
the cohesive mass of isolated cannabis trichomes is a substantially homogeneous cohesive mass of isolated cannabis trichomes.
the set of hashish product units comprises at least 10 hashish product units.

In one broad aspect, the present disclosure relates to a hashish product comprising a cohesive mass of isolated cannabis trichomes, the hashish product comprising a substantially homogeneous density throughout the cohesive mass thereof.

For example, in some implementations, the hashish product includes one or more of the following:
a segmentation of the cohesive mass into a plurality of portions results in a first portion having a respective density value which is substantially identical to an average density value of the plurality of portions.
about 85 percent or more, preferably about 90 percent or more, more preferably about 95 percent or more, of portions in the plurality of portions have a respective density value which is substantially identical to the average density value of the plurality of portions.
the respective physical characteristic value represents from about 0.9 to about 1.1 times, preferably from about 0.95 to about 1.05 times, more preferably from about 0.98 to about 1.02 times, the average density value of the plurality of portions.
the respective density is measured according to the following formula:
$$d e n s i t y = \frac{W a ∗ d w}{W a - W w}$$
wherein
   Wa = weight of the hashish portion in air,
   dw = density of water at 20° C.,
   Ww = weight of the hashish portion in water at 20° C.
the plurality of portions consists 12 portions, and wherein the average density is characterized with a variance of about 0.02% or less.
the hashish product has a density at about 20° C. > 1.00 g/cm³, preferably from about 1.02 g/cm³ to about 1.06 g/cm³.
the hashish product having a body comprising a network of internal voids.
comprising a cannabinoid content of from about 5 wt.% to about 90 wt.%, preferably from about 20 wt.% to about 90 wt.%.
the cannabinoid content comprises one or more of tetrahydrocannabinol (THC), cannabidiol (CBD), cannabigerol (CBG), cannabinol (CBN), and any combinations thereof.
the isolated cannabis trichomes are from one or more strain(s) of cannabis plant.
the hashish product further comprises one or more additional component.
the one or more additional component comprises one or more cannabinoid(s), one or more terpene(s), one or more flavonoid(s), water, one or more flavoring agent(s), one or more non-toxic coloring agent(s), or any combinations thereof.
the one or more additional components is incorporated within the cohesive mass of the hashish product.
the one or more additional components is distributed on at least a portion of a surface of the hashish product.
the cohesive mass is a substantially homogeneous cohesive mass of isolated cannabis trichomes.

In one broad aspect, the present disclosure relates to a hashish product comprising a cohesive mass of isolated cannabis trichomes, wherein a segmentation of the cohesive mass into a plurality of portions having identical respective mass volume results in a first portion having a respective weight value which is substantially identical to an average weight value of the plurality of portions.
about 85 percent or more, preferably about 90 percent or more, more preferably about 95 percent or more, of portions in the plurality of portions have a respective weight value which is substantially identical to the average density value of the plurality of portions.
the respective physical characteristic value represents from about 0.9 to about 1.1 times, preferably from about 0.95 to about 1.05 times, more preferably from about 0.98 to about 1.02 times, the average weight value of the plurality of portions.
comprising a cannabinoid content of from about 5 wt.% to about 90 wt.%, preferably from about 20 wt.% to about 90 wt.%.
the cannabinoid content comprises one or more of tetrahydrocannabinol (THC), cannabidiol (CBD), cannabigerol (CBG), cannabinol (CBN), and any combinations thereof.
the isolated cannabis trichomes are from one or more strain(s) of cannabis plant.
the hashish product further comprises one or more additional component.
the one or more additional component comprises one or more cannabinoid(s), one or more terpene(s), one or more flavonoid(s), water, one or more flavoring agent(s), one or more non-toxic coloring agent(s), or any combinations thereof.
the one or more additional components is incorporated within the cohesive mass of the hashish product.
the one or more additional components is distributed on at least a portion of a surface of the hashish product.
the cohesive mass is a substantially homogeneous cohesive mass of isolated cannabis trichomes.

Broadly stated, in some embodiments, the present disclosure relates to a process of making a hashish product. The method comprises: a) providing isolated cannabis trichomes, b) mixing the isolated cannabis trichomes under conditions sufficient to obtain a resinous mixture, and c) retrieving at least a portion of the resinous mixture through an extrusion die to obtain a hashish product comprising a substantially homogeneous cohesive mass of the isolated cannabis trichomes.

In some embodiments, the process includes one or more of the following features:
the isolated cannabis trichomes are kief.
the process further comprises cutting the hashish product according to a pre-established cutting operational parameter.
the cutting pattern includes cutting the hashish product along a transverse axis to obtain pieces thereof of identical length and/or weight.
the process further comprises incorporating one or more additional component prior to, simultaneously with, or following the mixing step.
the one or more additional component includes one or more cannabinoid, one or more terpene, one or more flavonoid, water, one or more flavoring agent, one or more non-toxic coloring agent, or a mixture thereof.
the one or more cannabinoid is in the form of a crude cannabis extract, a cannabis distillate, a winterized cannabis plant extract, cannabis rosin, cannabis resin, cannabis wax, cannabis shatter, or any combination thereof.
the one or more cannabinoid includes a plurality of cannabinoids such as tetrahydrocannabinol (THC), cannabidiol (CBD), cannabinol (CBN), or any combinations thereof.
the water is incorporated in the form of steam or liquid.
the mixing conditions includes a selected shear.
the mixing conditions further includes a pressure.
the pressure is between about 5 psi and about 500 psi.
the pressure is between about 20 psi and about 300 psi.
the conditions further includes mixing at a selected temperature.
the selected temperature is of about 140° C. or less.
the selected temperature is between about 20° C. and about 80° C.
the selected temperature is of about 60° C.
the mixing at the selected temperature is performed for a period of at least 5 minutes, or at least 10 minutes, or at least 15 minutes, or at least 20 minutes, or at least 25 minutes, or at least 30 minutes.
the mixing at the selected temperature is performed for less than 60 minutes, or less than 50 minutes, or less than 40 minutes, or less than 30 minutes.
the hashish product comprises a cannabinoid content of from about 5 wt.% to about 90 wt.%.
the hashish product comprises a cannabinoid content of up to about 60 wt.%.
the hashish product comprises a cannabinoid content of up to about 50 wt.%, or up to about 40 wt.%, or up to about 30 wt.%.
the isolated cannabis trichomes are from a single cannabis strain.
isolated cannabis trichomes are from a plurality of cannabis strains.
the mixing includes applying compression and shear forces to the isolated trichomes via a plurality of interpenetrate helicoidal surfaces within an elongated enclosure.
the elongated enclosure is an extruder device.
the interpenetrate helicoidal surfaces are on at least one screw, the method further comprising adjusting a rotational speed of the at least one screw within the elongated enclosure to obtain the cohesive mass.
the rotational speed of the at least one screw is between about 10 rpm and about 1000 rpm,
the rotational speed of the at least one screw is between about 15 rpm and about 500 rpm.

Broadly stated, in some embodiments, the present disclosure relates to a hashish product comprising a substantially homogeneous cohesive mass of isolated cannabis trichomes made by the process as described herein.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1A illustrates a non-limiting flowchart example of a process for obtaining a resinous mixture that is used to obtain an individual unit of hashish product in accordance with an embodiment of the present disclosure;

FIG. 1B illustrates a non-limiting flowchart example of a process for working the resinous mixture from FIG. 1A to obtain an individual unit of hashish product in accordance with an embodiment of the present disclosure;

FIG. 2 illustrates a non-limiting system implementing the method of FIG. 1A for manufacturing the hashish product unit;

FIG. 3 illustrates a non-limiting schematic of a setting for performing a density measurement;

FIG. 4 illustrates a non-limiting schematic for preparing samples from a hashish product manufactured by pressing;

FIG. 5 illustrates a non-limiting schematic for preparing samples from a hashish product manufactured according to an embodiment of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description to provide a thorough understanding of the invention. These details are provided for the purpose of non-limiting examples and the invention may be practiced according to the claims without some or all these specific details. Technical material that is known in the technical fields related to the invention has not been described in detail so that the invention is not unnecessarily obscured.

The present inventors have developed a hashish product and method of manufacturing same that addresses at least some of the above-identified problems.

For example, it has been observed that the hashish products of the present disclosure may present substantially consistent physical characteristics, which would be difficult to achieve with known techniques of the known prior art. Such consistent physical characteristics may allow, for example, obtaining products that are more uniform in terms of density per hashish product unit per se, or over multiple units (e.g., a set of units). Advantageously, it has been observed that such substantially consistent density allows manufacturers / end users to cut larger pieces of hashish product into smaller portions of pre-determined size and/or weight, thus ending up with consistent smaller portions in terms of dimensions and/or weight. This in turn, may also reduce / minimize quality control failures during large-scale manufacturing of the hashish product, e.g., quality control based on weight consistency of hashish products that are cut to have predetermined dimensions. which is advantageous in the context of large-scale industrial production. Such advantages may result in an increase in cost efficiency as well as improve inventory management due to reduction in waste.

Further, existing prior art procedures require each unit of hashish to be manually weighed and trimmed, resulting in non-uniform units and inconsistent appearance. The herein described process allows one to obtain substantially uniform and accurately weighted units since most, if not all, the process can be automated and implemented using mechanical means. Yet further, the processing conditions to form the hashish product can be consistently reproduced, thus allowing a product with substantially consistent composition and characteristics batch-over-batch.

Manufacturing of the hashish products as disclosed herein can advantageously be less messy and time-consuming than prior art hand-rolling procedures and reduce waste from product loss.

For example, it has been observed that the hashish products of the present disclosure may present substantially consistent homogeneity characteristics, which would be difficult to achieve with known techniques of the known prior art. Such homogeneity characteristics may allow, for example, improvement in the textural consistency, pliability and/or crumbliness of the hashish product. This in turn, may reduce / minimize quality control failures during large-scale manufacturing of the hashish product (e.g., quality control based on textural consistency, pliability and/or crumbliness). Advantageously, it has been observed that such hashish product may afford an enhanced and more consistent user experience in that the reduced crumbliness leads to better segmentation during use of the hashish product that results in reduction of waste material production. Further, it has been observed that implementing the method of manufacture herein described results in substantially fewer quality failures (e.g., based on textural consistency, pliability and/or crumbliness), which is advantageous in the context of large-scale industrial production. Such advantages may result in an increase in cost efficiency as well as improve inventory management due to reduction in waste.

Further, existing prior art procedures require each unit of hashish to be manually weighed and trimmed, resulting in non-uniform units and inconsistent appearance. The herein described process allows one to obtain substantially uniform and accurately weighted units since most, if not all, the process can be automated and implemented using mechanical means. Yet further, the processing conditions to form the hashish product can be consistently reproduced, thus allowing a product with substantially consistent composition and characteristics batch-over-batch.

Additionally, or alternatively, controlling the content and homogeneity of the herein described hashish products may allow the manufacturing of hashish products that contain substantially consistent isolated cannabis trichomes components content and distribution therein, within single product units and/or over multiple batches of product units. This in turn can be advantageous in view of increasing consumer demands for predictable user experience.

These and other advantages may become apparent to the person of skill in view of the present disclosure.

### Hashish Product

The hashish product of the present disclosure comprises a cohesive mass of isolated cannabis trichomes.

As used herein, the term “cannabis trichomes” generally refers to crystal-shaped outgrowths or appendages (also called resin glands) on cannabis plants typically covering the leaves and buds. Trichomes produce hundreds of known cannabinoids, terpenes, and flavonoids that make cannabis strains potent, unique, and effective.

As used herein, the term “isolated cannabis trichomes” refers to trichomes that have been separated from cannabis plant material using any method known in the art. The details of various methods for separating trichomes from the cannabis plant are well-known in the art. For example, and without wishing to be limiting in any manner, the isolated cannabis trichomes may be obtained by a chemical separation method or may be separated by manual processes like dry sifting or by water extraction methods. Solvent-less extraction methods can include mechanical separation of trichomes from the plant, such as sieving through a screen by hand or in motorized tumblers (see for example WO 2019/161509), or by submerging the cannabis plants in icy water (see for example US2020/0261824, which is herein incorporated by reference) and agitating to separate the trichomes from the plant and drying the trichomes. Because of inherent limitations to existing separation methods, some plant matter or other foreign matter can be present in isolated cannabis trichomes.

Isolated cannabis trichomes obtained by mechanical separation of trichomes from the cannabis plant biomass is typically referred to as “kief” (also “keef” or “kif”) and has a powdery appearance. The moisture content may be partially removed, often using heat and the finished kief is subsequently pressed or formed to obtain a hashish product. Typically, some residual plant material remains in the finished kief and thus in the resulting hashish product. In preferred embodiments of the present disclosure, the isolated cannabis trichomes is in the form of kief.

The isolated cannabis trichomes forming the hashish product of the present disclosure may originate from one or more than one strain of cannabis plant. It is known amongst consumers of hashish and other cannabis products that using isolated cannabis trichomes produced from more than one strain of cannabis plant allows a user to tune the psychoactive and/or entourage effect obtained by consuming the product. The mixing of cannabis plant strains may also allow to adjust the final concentration of a component of the product, for example but not limited to the cannabinoid content. Additionally, use of more than one strain allows for improved product and waste management - important in commercial production.

As used herein, the term “cannabis” generally refers to a genus of flowering plants that includes several species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. In terms of cannabinoids content, hemp has lower concentrations of tetrahydrocannabinol (THC) and higher concentrations of cannabidiol (CBD), which decreases or eliminates the THC-associated psychoactive effects.

The hashish product of the present disclosure comprises one or more cannabinoid(s). As used herein, the term “cannabinoid” generally refers to any chemical compound that acts upon a cannabinoid receptor such as CB1 and CB2. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBDA), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabionolic acid B (THCA-B), Δ9-tetrahydrocannabinolic acid A (THCA-A), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-l-yl)-5-pentyl-l,3-benzenediol); A4-cannabidiol (2-(6-isopropenyl-3-methyl-4-cyclohexen-l-yl)-5-pentyl-l,3-benzenediol); A3-cannabidiol (2-(6-isopropenyl-3-methyl-3-cyclohexen-l-yl)-5-pentyl-l,3-benzenediol); A3,7-cannabidiol (2-(6-isopropenyl-3-methylenecyclohex-l-yl)-5-pentyl-l,3-benzenediol); A2-cannabidiol (2-(6-isopropenyl-3-methyl-2-cyclohexen-l-yl)-5-pentyl-l,3-benzenediol); A1-cannabidiol (2-(6-isopropenyl-3-methyl-l-cyclohexen-l-yl)-5-pentyl-l,3-benzenediol); and A6-cannabidiol (2-(6-isopropenyl-3-methyl-6-cyclohexen-l-yl)-5-pentyl-l,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

In one embodiment, the hashish product of the present disclosure contains the one or more cannabinoid(s) in an amount sufficient for the user to experience a desired effect when consuming the product. For example, the hashish product may comprise from about 5 wt.% to about 90 wt.% cannabinoid or any value therebetween, or in a range of values defined by any values therebetween. For example, the hashish product may comprise up to about 90 wt.%, up to about 80 wt.%, up to about 70 wt.%, up to about 60 wt.%, or up to about 50 wt.%, or up to about 40 wt.%, or up to about 30 wt.% or any value therebetween, or in a range of values defined by the aforementioned values. In another embodiment, the hashish product may include up to 1000 mg THC per hashish product unit, depending on specific implementations of the present disclosure.

In some embodiments, alternatively or additionally, the hashish product of the present disclosure may include one or more cannabinoid other than THC, such as for example, delta-8-THC, CBD, CBN, and the like, or any combinations thereof, in similar or different amounts, as well as one or more terpenes, depending on specific implementations of the present disclosure.

A cannabinoid may be in an acid form or a non-acid form, the latter also being referred to as the decarboxylated form since the non-acid form can be generated by decarboxylating the acid form.

In some embodiments, the hashish product of the present disclosure has a substantially homogeneous cohesive mass of isolated trichomes.

By “substantially homogeneous”, it is meant that the hashish product has a constant or uniform distribution of isolated trichomes throughout its cohesive mass. In other words, the manufacturing procedures described herein process the isolated trichomes in such fashion to result in mixing thereof into a substantially constant, uniform cohesive mass.

### Additional Components

The hashish product according to the present disclosure may also comprise one or more additional components.

In some embodiments, the one or more additional components may be added to alter the characteristics of the hashish product, such as cannabinoid content, potency, entourage effect, odor, color, consistency, texture, pliability, and the like.

In some embodiments, the one or more additional components may be incorporated throughout the hashish product, or the one or more additional components may be distributed on at least a portion of a surface of the hashish product, for example as a coating. In some embodiments, the one or more additional components may be substantially homogeneously distributed on the at least portion of the surface of the hashish product. By “substantially homogeneously distributed”, it is meant that the amount of the one or more additional component is uniform on the at least portion of the surface of the hashish product.

The one or more additional components may be any suitable food grade and/or non-toxic composition or component known in the art. As will be recognized by those of skill in the art, the toxicity of each type of additional component may be dependent on the method of consumption of the hashish product. For example, in applications where smoke / vapor produced by the hashish product is to be inhaled, suitable additional components may include, but are not limited to one or more cannabinoid, one or more terpene (also referred to herein as a “terpene blend”), one or more flavonoid, water, or any combination thereof.

The one or more additional components may be a cannabinoid. The cannabinoid may be extracted from any suitable source material including, but not limited to, cannabis or hemp plant material (e.g., flowers, seeds, and trichomes) or may be manufactured artificially (for example cannabinoids produced in yeast, as described in WO WO2018/148848). Cannabinoids can be extracted from a cannabis or hemp plant material according to any procedure known in the art. For example, and without wishing to be limiting, a “crude extract” containing a cannabinoid may be obtained by extraction from plant materials using for example aliphatic hydrocarbons (such as propane, butane), alcohols (such as ethanol), petroleum ether, naphtha, olive oil, carbon dioxide (including supercritical and subcritical CO₂), chloroform, or any combinations thereof. Optionally, the crude extract may then be “winterized”, that is, extracted with an organic solvent (such as ethanol) to remove lipids and waxes (to produce a “winterized extract”), as described for example in US 7,700,368, US 2004/0049059, and US 2008/0167483, which are each herein incorporated by reference in their entirety. Optionally, the method for obtaining the cannabinoid may further include purification steps such as a distillation step to further purify, isolate or crystallize one or more cannabinoids, which is referred to in the art and herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be cut with one or more terpenes. The crude extract, the winterized extract or the distillate may be further purified, for example using chromatographic and other separation methods known in the art, to obtain an “isolate”. Cannabinoid extracts may also be obtained using solvent-less extraction methods; for example, cannabis plant material may be subjected to heat and pressure to extract a resinous sap (“rosin”) containing cannabinoids; methods for obtaining rosin are well-known in the art.

The one or more additional components may be a terpene. As used herein, the term “terpene” generally refers to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Any suitable terpene may be used in the hashish product of the present invention. For example, terpenes originating from cannabis plant may be used, including but not limited to aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, hashishene, and derivatives thereof. Further examples of terpenes are discussed in US Patent Application Pub. No. US2016/0250270, which is herein incorporated by reference in its entirety for all purposes. The hashish product of the present disclosure may contain one or more terpene(s). The one or more terpene(s) may originate from the hashish, from an additional component, or both. In some embodiments, the hashish product of the present disclosure may include the one or more terpene(s) in an amount (the “terpene content”) sufficient for the user to experience a desired entourage effect when consuming the product. For example, the hashish product may comprise from about 0.5 wt.% to about 15 wt.% terpene, for example up to about 15 wt.%, or up to about 10 wt.%, or up to about 5 wt.%, or up to about 4 wt.%, or up to about 3 wt.%, or up to about 2 wt.%, or up to about 1 wt.%. For example, the one or more terpene(s) may include hashishene. Without wishing to be bound by theory, hashishene is believed to be a terpene produced by rearrangement of myrcene that may be found in hashish after mechanical processing, and that may be responsible for the typical desirable “hashish flavour”.

The one or more additional components may be a flavonoid. The term “flavonoid” as used herein refers to a group of phytonutrients comprising a polyphenolic structure. Flavonoids are found in diverse types of plants and are responsible for a wide range of functions, including imparting pigment to petals, leaves, and fruit. Any suitable flavonoid may be used in the hashish product of the present invention. For example, flavonoids originating from a cannabis plant may be used, including but not limited to: apigenin, cannflavin A, cannflavin B, cannflavin C, chrysoeril, cosmosiin, flavocannabiside, homoorientin, kaempferol, luteolin, myricetin, orientin, quercetin, vitexin, and isovitexin.

The reader will readily understand that in some embodiments, the one or more components may include any combinations of the herein described one or more component(s).

### Uniform Physical Characteristics

The hashish product of the present disclosure has substantially uniform physical characteristics.

For example, such uniform physical characteristics may characterize a hashish product unit per se. For example, the herein described uniform physical characteristics may be assessed intra-product, i.e., within a portion of a single product unit and compared to the average corresponding physical characteristics throughout the same single product. For example, the herein described uniform physical characteristics may be assessed inter-product, in a first product and compared to the corresponding physical characteristics of a second product.

For example, such uniform physical characteristics may characterize a set of hashish product units per se. For example, the herein described uniform physical characteristics may be assessed intra-batch, i.e., within one or more product units within a set of a plurality of hashish product units and compared to the average of the corresponding physical characteristics in the set of hashish product units. For example, the herein described uniform physical characteristics may be assessed inter-batch, i.e., within a first set having a first plurality of hashish product units and the average value for the physical characteristics compared to the corresponding average value for the physical characteristics assessed within a second set having a second plurality of hashish product units.

In some embodiments, the hashish product of the present disclosure has a cohesive mass of isolated cannabis trichomes, wherein a segmentation of the cohesive mass into a plurality of portions having identical respective mass volume results in a first portion having a respective weight value which is substantially identical to an average weight value of the plurality of portions.

In some embodiments, the hashish product of the present disclosure has a cohesive mass of isolated cannabis trichomes, wherein the hashish product comprises a substantially homogeneous density throughout the cohesive mass thereof.

In some embodiments, the hashish product of the present disclosure has a cohesive mass of isolated cannabis trichomes, wherein a segmentation of the cohesive mass into a plurality of portions having identical respective mass volume results in a first portion having a respective weight value which is substantially identical to an average weight value of the plurality of portions, and wherein the hashish product comprises a substantially homogeneous density throughout the cohesive mass thereof.

In some embodiments, a set of hashish product units of the present disclosure is characterized with each hashish product unit in the set of hashish product units comprising a cohesive mass of isolated cannabis trichomes, and wherein a first hashish product unit in the set of hashish product units is characterized with a physical characteristic which is substantially identical to an average of the physical characteristic in the set of hashish product units.

In some implementations, the physical characteristic which is substantially identical to an average of the physical characteristic in the set of hashish product units is a cross section dimension of the hashish product unit. For example, a respective cross section dimension can be substantially identical to the corresponding average cross section dimension in the set of hashish product units. In such embodiments, the set of hashish product units includes one or more of the following: about 85 percent or more of hashish product units in the set of hashish product units have a respective cross section dimension which is substantially identical to the corresponding average cross section dimension in the set of hashish product units; about 90 percent or more of hashish product units in the set of hashish product units have a respective cross section dimension which is substantially identical to the corresponding average cross section dimension in the set of hashish product units; about 95 percent or more of hashish product units in the set of hashish product units have a respective cross section dimension which is substantially identical to the corresponding average cross section dimension in the set of hashish product units; the respective cross section dimension represents from about 0.9 to about 1.1 times the corresponding average cross section dimension in the set of hashish product units; the respective cross section dimension represents from about 0.95 to about 1.05 times the corresponding average cross section dimension in the set of hashish product units; the respective cross section dimension represents from about 0.98 to about 1.02 times the corresponding average cross section dimension in the set of hashish product units.

In some implementations, the physical characteristic which is substantially identical to an average of the physical characteristic in the set of hashish product units is the respective unit weight. For example, a respective unit weight can be substantially identical to the average unit weight in the set of hashish product units. In such embodiments, the set of hashish product units includes one or more of the following: about 85 percent or more of hashish product units in the set of hashish product units have a respective unit weight which is substantially identical to the average unit weight in the set of hashish product units; about 90 percent or more of hashish product units in the set of hashish product units have a respective unit weight which is substantially identical to the average unit weight in the set of hashish product units; about 95 percent or more of hashish product units in the set of hashish product units have a respective unit weight which is substantially identical to the average unit weight in the set of hashish product units; the respective unit weight represents from about 0.9 to about 1.1 times the average unit weight in the set of hashish product units; the respective unit weight represents from about 0.95 to about 1.05 times the average unit weight in the set of hashish product units; or the respective unit weight represents from about 0.98 to about 1.02 times the average unit weight in the set of hashish product units.

In some implementations, the physical characteristic which is substantially identical to an average of the physical characteristic in the set of hashish product units is a density of the hashish product unit. For example, a respective density measured at 20° C. can be substantially identical to the average density measured at 20° C. in the set of hashish product units. In such embodiments, the set of hashish product units includes one or more of the following: about 85 percent or more of hashish product units in the set of hashish product units have a respective density measured at 20° C. which is substantially identical to the average density measured at 20° C. in the set of hashish product units; about 90 percent or more of hashish product units in the set of hashish product units have a respective density measured at 20° C. which is substantially identical to the average density measured at 20° C. in the set of hashish product units; about 95 percent or more of hashish product units in the set of hashish product units have a respective density measured at 20° C. which is substantially identical to the average density measured at 20° C. in the set of hashish product units; the respective density measured at 20° C. represents from about 0.9 to about 1.1 times the average density measured at 20° C. in the set of hashish product units; the respective density measured at 20° C. represents from about 0.95 to about 1.05 times the average density measured at 20° C. in the set of hashish product units; or the respective density measured at 20° C. represents from about 0.98 to about 1.02 times the average density measured at 20° C. in the set of hashish product units.

The reader will readily understand that in some implementations, the physical characteristic may include one or more of those characteristics described herein.

Such consistency in terms of physical characteristics obtained with the hashish products of the present disclosure affords an unexpected technical advantage. For example, it was unexpected in that an equivalent consistency in terms of physical characteristics was not obtained in hashish products made with prior art pressing techniques. Indeed, the present inventors surprisingly discovered that large scale production of hashish products made with prior art pressing techniques suffered from significant variations in terms of physical characteristics, such as density, weight, and / or dimensions. Such variability can be detrimental for a number of reasons. For example, in some cases, consumers may wish to introduce a hashish product into a smoking device having a smoking receptacle of fixed dimension - hashish products with variable density would lead to variable weights for a given volume, thus resulting in variable amounts of hashish material in the smoking receptacle of fixed dimension from one use to another. Conversely, having a hashish product with consistency in terms of physical characteristic as described herein will allow a more consistent user experience in that substantially identical amounts of hashish product will fit in the smoking receptable from one hashish use to another. As the amount of hashish fitted into the smoking receptacle determines the amount of cannabinoid(s) consumed, consistency in terms of amounts of hashish will correlate with consistency in terms of cannabinoids consumed and resulting user experience. Other advantages will become apparent to the reader in view of the teachings herein.

### Methods of Using Hashish

Hashish products are typically used for recreational or medicinal purposes. For example, hashish can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered and rate and time-course of administration will depend on the nature and severity of the disease or condition being treated and typically also takes into consideration the condition of the individual subject, the method of administration and the like.

### Manufacturing Process

The hashish product may be produced by mechanically mixing the components thoroughly to provide a substantially homogeneous resinous mixture. By the term “mechanically mixing” or “mechanical mixing”, it is meant mixing using any suitable mechanical means. The mechanical means may be a plurality of interpenetrate helicoidal surfaces within an elongated enclosure or barrel, a non-limiting example of which is an extruder apparatus.

An extruder is a machine used to perform the extrusion process. Manufacturing by extrusion occurs when a material (usually pellets, dry powder, rubber, plastic, metal bar stock or food) is heated and pushed through a die assembly. A die is a mold that shapes the heated material as it is forced through a small opening from the inside of the extruder to the outside. Using a system of barrels or cylinders containing interpenetrate helicoidal surfaces, e.g., screw pumps or extruder screws, the extruder can mix the ingredients while heating and propelling the extrudate through the die to create the desired shape.

An extruder can have a single extruder screw or twin extruder screws, and can be configured to have one or more mixing zones, one or more temperature zones, and one or more input zones. The input zones are used for introduction of material. The mixing zones apply compression and shear forces to the input materials, blending until they are homogenized. The extruder die assembly may perform a variety of functions: it may form or shape the extrudate, it may divide the extrudate into multiple extrudates, it may inject one or more component into the extrudate, and it may compress and reduce the cross-sectional area of the extrudate.

Single screw extruders are known in the art - the screws of such extruders comprise grooves and may be cylindrical, conical, tapered and the likes as described for example in CA 2,731,515, US 6,705,752, CN101954732 and CN201792480, where each of which is herein incorporated by reference in its entirety. Twin screw extruders are also known in the art - screws of such extruders may be parallel or non-parallel, converging or non-converging, with or without differential speed, counter or non-counter rotating as described for example in US 6,609,819, WO 2020/220390, WO 2020/220495 and US 2010/0143523, where each of which is herein incorporated by reference in its entirety. Single screw and twin screw arrangements may also be integrated within a single extruder device, as described for example in US 10,124,526, which is herein incorporated by reference in its entirety. It will be readily appreciated that extruders have flexible configuration (in terms of mixing zones, temperature zones, input zones, etc.) and that any suitable configuration of the extruder apparatus that produces the hash product may be used within the context of the present disclosure.

The mechanical mixing can be applied to the isolated cannabis trichomes within the extruder under conditions sufficient to obtain a cohesive, continuous, and substantially homogenous resinous mixture. The conditions or variables that can be modified during production are discussed later in this text.

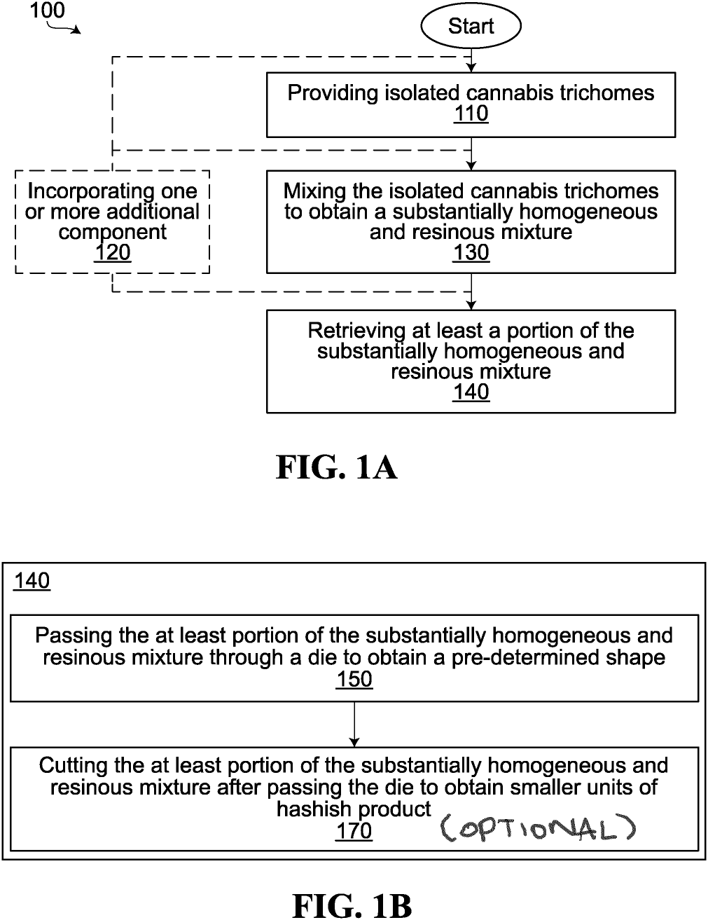

FIG. 1A is a flowchart of a general process 100 of making individual units of hashish product in accordance with an embodiment of the present disclosure.

The process 100 includes a step 110 of providing isolated cannabis trichomes (alone or together with one or more additional components as will be described later in this text). In one embodiment, the isolated cannabis trichomes may include trichomes isolated from a single cannabis strain. In another embodiment, the isolated cannabis trichomes may include trichomes isolated from a plurality of cannabis strains, which may have different respective cannabinoid(s) and/or terpene(s) concentrations. Both are viable options, the choice of one over the other may be driven by practical considerations, such as inventory management consideration and/or cannabinoid content of the hashish product, or by the desired user experience. It is known amongst consumers of hashish and other cannabis products that using isolated cannabis trichomes produced from more than one strain of cannabis plant may allow a user to tune the psychoactive and/or entourage effect obtained by consuming the product. The mixing of cannabis plant strains may also allow adjustments to the final concentration of one or more components of the product, for example but not limited to the cannabinoid content. Additionally, use of more than one strain allows for improved product and waste management - important in commercial production. The isolated cannabis trichomes can be kief.

The isolated cannabis trichomes are mixed under conditions sufficient to obtain a substantially homogenous and resinous mixture at step 130. For example, such conditions may include shear and pressure, and optionally temperature, which may be varied to alter the characteristics of the hashish product. Such characteristics may include, but without being limited to, stiffness (i.e., characteristic that defines the level of malleability of the hashish product), hardness or resistance to localized deformation (i.e., characteristic that determines how easy it is to cut or separate the hashish product), toughness (i.e., characteristic that determines the likelihood that the hashish product deforms rather than fractures under an applied force), color, tactual characteristics, and the like.

In some examples, the conditions may include performing the mixing of the isolated cannabis trichomes (and optionally the one or more components, when present) at a selected temperature. The selected temperature can be for example but without being limited to a temperature of about 140° C. or less, such as any temperature of from about 20° C. to about 120° C. For example, a temperature of about 20° C., about 30° C., about 40° C., about 50° C., about 60° C., about 70° C., about 80° C., about 90° C., about 100° C., about 110° C., or about 120° C. In some embodiments, the mixing at the selected temperature is performed for a period of at least 5 minutes, or at least 10 minutes, or at least 15 minutes, or at least 20 minutes, or at least 25 minutes, or at least 30 minutes. For example, the mixing at the selected temperature can be performed for less than 60 minutes, or less than 50 minutes, or less than 40 minutes, or less than 30 minutes.

In some examples, the conditions may include submitting the isolated trichomes to pressure while mixing and/or heating at values of from about 5 psi to about 250 psi, including any ranges therein or any value therein. For example, the pressure may include a pressure in the range of about 0.5 to about 15 US tons, or about 1 to about 15 US tons, or about 1 to about 10 US tons, or about 2 to about 8 US tons, or any value within such ranges. For example, the pressure may be between about 5 psi and about 500 psi, such as between about 20 psi and about 300 psi, including any values there in-between. For example, a pressure of about 20 psi, about 30 psi, about 40 psi, about 50 psi, about 100 psi, about 150 psi, about 200 psi, about 250 psi, about 300 psi. The person of skill will readily understand that a given pressure value may be selected depending on the die that is used to form the hashish product, as described elsewhere in this text. Additionally, a non-limiting example, the pressure may be performed for a time ranging from about 0.5 (30 seconds) to about 30 minutes, including any value within such range. For example, a period of about 20 minutes, about 10 minutes, about 5 minutes, about 4 minutes, or less.

In a practical implementation, the mixing includes applying compression and shear forces to the isolated cannabis trichomes via a plurality of interpenetrate helicoidal surfaces within an elongated enclosure. Preferably, the elongated enclosure is an extruder device having at least one screw. The mixing shear and compressive forces can be controlled by modulating the rotational speed of at least one of the screws within the extruder. In such embodiments, the extruder screw rotation per minute (rpm) can be selected to perform the mixing step 130 at a value of for example from about 10 to about 1000 rpm, such as from about 15 to about 500 rpm, or from about 25 to about 450 rpm, or from about 30 to about 400 rpm, or from about 45 to about 450 rpm including any value within any of these ranges. In such embodiment, the pressure applied by the extruder screw can be accompanied by heat to enhance mixing of the isolated cannabis trichomes, extract the resinous content of the trichomes and obtain a heated, cohesive, continuous and substantially homogenous resinous mixture. In such embodiment, the heating and mixing can continue until a desired level of homogeneity is obtained. For example, for a minimum time of about 30 minutes, about 20 minutes, about 10 minutes, or less. In some embodiments, the heating and mixing continues until the desired level of homogeneity is determined by testing samples of mass retrieved from the process.

In embodiments where the heating and mixing are performed in a single screw extruder, the residence time within the extruder barrel is directly related to the length of the barrel and the rotational speed of the single screw. To increase mixing time of the components within the barrel, the components can travel through the length of the barrel, and then be redirected to the inlet (rather than proceed through the die).

Optional step 120 includes incorporating one or more additional component at one or more steps during the process 100. For example, one or more additional component can be added to the isolated trichomes prior to, simultaneously with, or following step 110, or prior to, simultaneously with, or following the mixing step 130. Multiple additional components may be added in a single step or may be added separately in one or more consecutive steps or at different times or points along the process 100. The one or more additional components can be one or more cannabinoids, one or more terpenes, one or more flavonoids, water, one or more flavoring agents, one or more non-toxic coloring agents, or any combination thereof. The person of skill will readily appreciate that water could be added in the form of steam, liquid or in combination. When the one or more component comprises a cannabinoid, the cannabinoid may be provided in the form of a cannabis extract (including a crude extract, or a winterized extract), a distillate, an isolate, cannabis rosin, cannabis resin, cannabis wax, cannabis shatter.

In some embodiments, the one or more additional component may be incorporated during the process and thus result in the one or more additional component being incorporated within the hashish product. Alternatively, or additionally, the one or more additional component may be distributed on at least a portion of a surface of the hashish product, for example as a coating. In some embodiments, the coating may completely cover the surface of the hashish product.

In some embodiments, the one or more cannabinoids can be in extracted and purified form and may include a crude cannabis extract, a cannabis distillate, a cannabis isolate, a winterized cannabis extract, cannabis rosin, cannabis resin, cannabis wax, or cannabis shatter, or any possible combination thereof.

In some embodiments, the one or more terpenes may include one or more terpenes which are endogenous to the cannabis strain or plurality of cannabis strains from which stem the isolated cannabis trichomes. The one or more terpenes may include one or more terpenes that are not naturally found in the one or more cannabis strain(s) from which stem the isolated cannabis trichomes.

Once the substantially homogenous and resinous mixture is obtained at step 130, at least a portion of the substantially homogenous and resinous mixture is retrieved at step 140 to obtain an individual unit of hashish product having a cohesive mass of the isolated trichomes.

FIG. 1B includes additional sub-steps of step 140, which may be implemented if desired. For example, the portion of the substantially homogenous and resinous mixture can be passed through a die at step 150, which may be configured to impart a pre-determined shape to the resinous mixture. Finally, the solid or semi-solid hashish product from step 150 can be cut at optional step 170 according to a pre-determined cutting pattern, a pre-determined weight, or a pre-determined length to obtain smaller units of hashish product for a pre-determined packaging size.

There are several options to implement the herein described process 100.

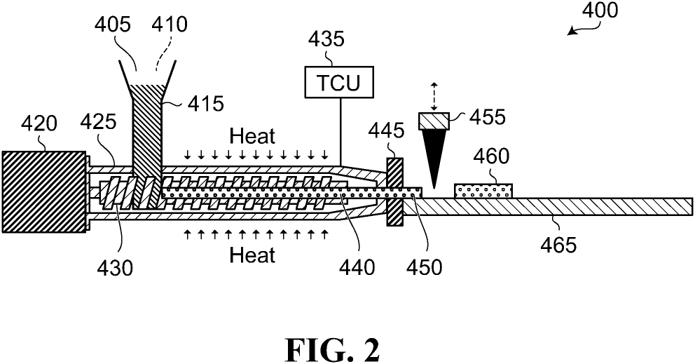

FIG. 2 illustrates a system 400 for implementing the process 100 to make individual units of hashish product 460 in accordance with an embodiment of the present disclosure. Other embodiments will be readily envisioned by the person of skill in view of the present disclosure. The system 400 includes an extruder apparatus 425 that uses mechanical mixing means to amalgamate the isolated cannabis trichomes 405 (and optionally one or more additional component(s) 410) into a coherent and substantially homogenous cohesive mass 450.

In this embodiment, the system 400 further comprises an optional feed input 415 through which the isolated cannabis trichomes 405 (and optionally the one or more additional component(s) 410) can be fed. As discussed previously, non-limiting examples of such one or more additional component(s) 410 include terpenes, flavonoids, water in the form of steam or liquid, cannabinoids in the form of crude extracts, distillates, isolates, winterized cannabis extracts, rosin, shatter, resins, or any combinations thereof. In another embodiment, at least a portion of the one or more additional component(s) 410 may be fed into the extruder apparatus 425. Other embodiments will be readily envisioned by the person of skill in view of the present disclosure, where for example the feed input 415 may be located elsewhere along the extruder apparatus 425 body or more than one input feed input may be located along the extruder apparatus 425, for example to feed the one or more additional component(s) 410.

The extruder apparatus 425 is powered by a motor 420 that drives at least one extruder screw 430 to apply pressure and mechanical shear on the isolated cannabis trichomes 405 (and optionally the one or more additional component(s) 410) entering the extruder 425. For example, the extruder screw 430 may be configured for applying compression and shear forces to the isolated cannabis trichomes 405 via a plurality of interpenetrate helicoidal surfaces present along at least a portion of the extruder screw 430.

When desired, the system 400 may also implement heating, such as within one or more predetermined portions (each a “heating zone”) of the extruder apparatus 425, or throughout the length of the extruder apparatus 425, depending on specifics applications. The operating parameters of the extruder apparatus 425, such as those discussed previously (e.g., the heating temperature, pressure, and extruder screw rotation per minute (rpm)), can be selected to alter residence time of the resinous mixture 440 (or isolated cannabis trichomes 405) in the extruder apparatus 425 to obtain the cohesive mass 450. Advantageously, it has been observed that operating parameters such as heat and extrusion speed change the pressure experienced at the die and may alter the characteristics of the hash product discussed above.

In some embodiments, the heating may additionally advantageously assist in homogeneous mixing of the isolated cannabis trichomes and optional additional components to form the cohesive mass 450.

In some embodiments, the heating time may be of at least 2 minutes, for example, at least 5 minutes, at least 10 minutes, at least 15 minutes, at least 20 minutes, at least 25 minutes, at least 30 minutes, at least 40 minutes, at least 60 minutes, depending on the specifics of an application, in each of the one or more heating zones of the extruder apparatus 425.

In some embodiments, the pressure applied by the extruder screw 430 is accompanied by heat to enhance mixing of the batch of isolated cannabis trichomes 405 (and optionally the one or more additional component(s) 410), extract the resinous content of the isolated cannabis trichomes and obtain a heated, cohesive, continuous, and substantially homogenous resinous mixture 440.

In some embodiments, the heat may be applied through a heating element (not shown) that is embedded with the extruder screw 430 and extends along the entire or part(s) of the length of the extruder screw 430. In another embodiment, the heat may be applied through a heated jacket (not shown) that partially, or entirely, surrounds the extruder apparatus 425. To control the amount of heat input to the extruder and ensure that the quality of the resinous mixture 440 would not be compromised, a temperature controlling unit (TCU) 435 can also be associated with the extruder apparatus 425 to monitor heat within the extruder apparatus 425 and take any necessary action in the event of major deviations from the intended extrusion temperature.

For example, the temperature controlling unit (TCU) 435 may include a thermometer (not shown) that is connected to the exterior body of the extruder with its distal end in contact with the resinous mixture 440 recording an average resinous mixture temperature (T1). In another embodiment, the thermometer may be connected to the exterior body of the extruder apparatus 425 with its distal end attached to the outer surface of the extruder apparatus 425 recording an average operating temperature (T2) wherein T2= T1 -ΔT with ΔT being a temperature offset.

The resinous mixture 440 then exits the extruder apparatus in the form of an elongated, continuous solid or semi-solid cohesive mass 450. Optionally, the extrusion apparatus 425 may include a die 445 at the outlet thereof, which may impart any pre-determined shape to the cohesive mass 450. At that point in the process, the long and continuous solid or semi-solid cohesive mass 450 can be subjected to ambient temperature and pressure.

A cutting means 455 may be placed downstream of the extruder die 445. The cutting means 455 may be configured to cut the cohesive mass 450 according to a pre-established cutting pattern. In a non-limiting example of implementation, the pre-established cutting pattern may comprise cutting the cohesive mass 450 along a transverse axis and at pre-determined time intervals so as to obtain hashish product unit 460 of a pre-determined length and/or weight. For example, to obtain a plurality of hashish product units 460 with consistent dimensions and/or weight, the cutting means 455 can act intermittently to cut the cohesive mass 450 into individual units of hashish product 460. The individual units of hashish product 460 could be further transferred onto a flat conveyor belt 465 or fall under gravity over an inclined conveyor belt (not shown) and sent for packaging and/or storage.

### Density Determination

Density of solid materials such as a hashish product can be determined according to different methods and via different apparatus and according to different standards known in the art.

For solid materials with regular shapes and/or neat geometries, density can be determined by dividing the mass of the solid material by its volume. As a person skilled in the art would appreciate, for solid materials with irregular shapes and/or uneven surfaces, indirect methods may be used. In some cases, for example, the mass of the product sample can be separately determined with a balance while sample volume is determined by immersing the sample in a liquid container to determine volume of the product based on the volume of liquid that is displaced upon immersing the sample. In some other cases, for example, density can be calculated according to ASTM D1505 using a density column filled with liquid while reference balls with known densities are floated in the liquid column and the sample density can be determined based on its floating position in the density column and relative to the reference balls. In some other case, density can be calculated according to ASTM D792 using a density determination kit equipped with a balance to determine the mass of the sample both in the air and immersed conditions wherein the sample is immersed in a liquid of known density. Such density determination devices are based on hydrostatic weighting and Archimedean principle and are known to a person skilled in the art. Different models may be available including but not limited to Sartorius AG Density Determination Kit (models YDK03MS and YDK04MS), MettlerToledo Density Determination Devices (available in the USA), and the like.

In one preferred embodiment, density determination is performed using a Sartorius Density Determination Kit (model YDK03MS or YDK04MS).

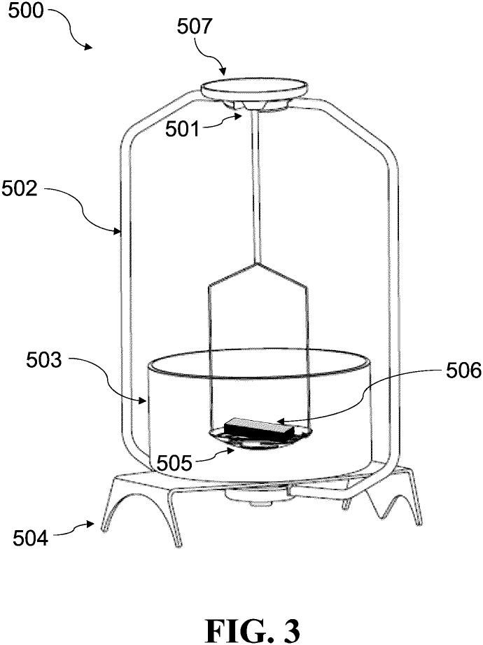

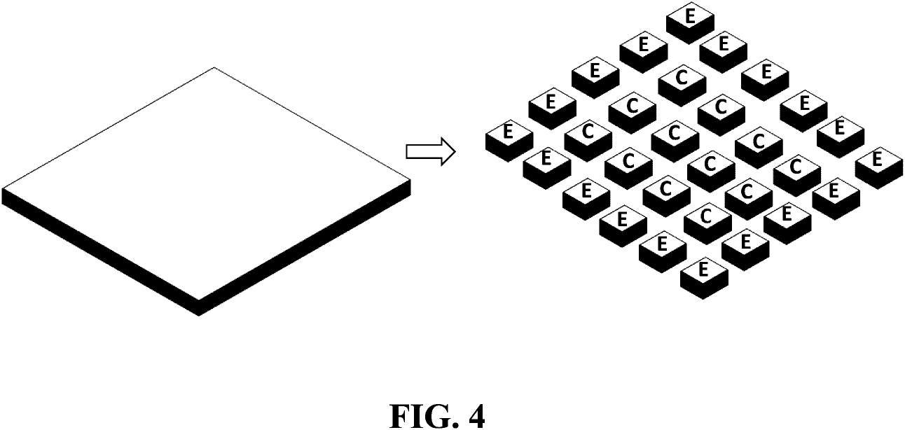

FIG. 3 a non-limiting example of a Sartorius device 500 employed to determine density of a sample of hashish product 506. The device for measuring density comprises a sample holder 505 fixed onto a hanger assembly 501 which is in turn supported by a bar frame 502. The sample holder 505 holding the hashish product sample 506 is immersed in a beaker 503 filled with a liquid (typically water - not shown). A thermometer may also be placed inside the beaker (not shown) to measure liquid temperature inside the beaker. The beaker 503 is placed on a metal support plate 504. The density of the hashish product sample 506 is then determined

The “Density Test” consists of the following: weight of the hashish product sample 506 is determined both in air and in the liquid (immersed condition) using the hydrostatic balance 507, and then, the hashish product density density is calculated according to the following formula:

$$d e n s i t y = \frac{W a ∗ d w}{W a - W w}$$

wherein
Wa = weight of the hashish product sample in air,
dw = density of water at 21° C.,
Ww = weight of the hashish product sample in water at 21° C.

### EXAMPLES

The following examples describe some exemplary modes of making and practicing certain compositions that are described herein. These examples are for illustrative purposes only and are not meant to limit the scope of the compositions and methods described herein.

### Example 1 - Extruding Hashish

In this example, hashish products were manufactured with isolated trichomes from NLxBB cannabis strain in accordance with an embodiment procedure of the present disclosure.

A batch of 270 g of dried isolated trichomes was mixed thoroughly and placed into the hopper of an ETPI Lab extruder (The Bonnot Company, USA). The extruder was operated at a temperature of 60° C. and screw speed of 15 RPM. The processing time for the batch was 5 minutes. A hashish product having a cohesive mass of isolated trichomes was retrieved through an extrusion die of the extruder.

### Example 2 - Extruding Hashish - Varying Parameters to Influence Cohesion

In this example, hashish products were manufactured with isolated trichomes from NLxBB or Grace cannabis strains in accordance with an embodiment procedure of the present disclosure.

Batches of from 125 g to 230 g of dried isolated trichomes were mixed thoroughly and respectively placed into the hopper of an ETPI Lab extruder equipped with a round die while varying several process parameters, as reproduced in Table 1.

TABLE 1

| Die | Strain | Temp (°C) | Screw Speed (rpm) | Pressure (PSI) | Cohesion Assessment |
|---|---|---|---|---|---|
| 24 x 3 mm round | NLxBB | 60 | 25 | 0-1 | No cohesion |
| 24 x 3 mm round | NLxBB | 60 | 45 | 3-8 | No cohesion |
| 24 x 3 mm round | NLxBB | 60 | 25 | 0-1 | No cohesion |
| 24 x 3 mm round | NLxBB | 60 | 45 | 3-8 | No cohesion |
| 24 x 3 mm round | NLxBB | 80 | 25 | 0-3 | No cohesion |
| 24 x 3 mm round | NLxBB | 80 | 45 | 0-2 | No cohesion |
| 24 x 3 mm round | NLxBB | 80 | 25 | 0-2 | No cohesion |
| 24 x 3 mm round | NLxBB | 80 | 45 | 0-2 | No cohesion |
| 24 x 3 mm round | NLxBB | 35 | 45 | 0-20 | Some cohesion |
| 24 x 3 mm round | Grace | 20 | 25 | 20-50 | Some cohesion |
| 24 x 3 mm round | Grace | 20 | 45 | 50-80 | Some cohesion |
| 24 x 3 mm round | Grace | 30 | 25 | 20-50 | Good cohesion |
| 24 x 3 mm round | Grace | 40 | 25 | 0-20 | Good cohesion |
| 24 x 3 mm round | Grace | 50 | 25 | 0-10 | Average cohesion |
| 24 x 3 mm round | Grace | 40 | 45 | 0-20 | Average cohesion |
| 38 x 2 mm round | Grace | 50 | 45 | 10-30 | Very good cohesion and texture |
| 38 x 2 mm round | Grace | 40 | 45 | 90-140 | Excellent cohesion and texture |
| 38 x 2 mm round | Grace | 35 | 45 | 180-220 | Very good cohesion and texture |
| 38 x 2 mm round | Grace | 70 | 45 | 0-10 | No cohesion |
| 38 x 2 mm round | Grace | 50 | 45 | 15-30 | Good cohesion |

Table 1 shows that hashish cohesion obtained with an extruder is an interplay between operating temperature, extruder screw speed and pressure. Table 1 also shows that the extruder screw speed, within the range in this example, has less impact in achieving an acceptable cohesion in the hash product. In specific embodiments, operating temperatures in the range of 35° C. to 50° C. and/or pressures between about 90 to about 220 PSI result in a product having more cohesion.

### Example 3 - Extruding Hashish - Varying Parameters to Influence Hardness / Pliability

In this example, hashish products were manufactured with isolated trichomes from Grace, SL or NLxBB cannabis strains in accordance with an embodiment procedure of the present disclosure.

Batches of from 120 g to 300 g of dried isolated trichomes were mixed thoroughly and respectively placed into the hopper of an ETPI Lab extruder equipped with a 20 mm by 5 mm rectangular die while varying several process parameters, as reproduced in table 2.

TABLE 2

| Strain | Temp (°C) | Screw Speed (rpm) | Pressure (PSI) | Assessment |
|---|---|---|---|---|
| Grace | 40 | 10 | 300 | Hard, Good cohesion |
| Grace | 50 | 10 | 300 | Hard, Good cohesion |
| Grace | 60 | 15 | 80-110 | Hard, Good cohesion |
| SL | 60 | 15 | 80-130 | Hard, Very good cohesion |
| SL | 70 | 15 | 80-130 | Hard, Very average cohesion |
| NLxBB | 60 | 15 | 150 | Hard, Very good cohesion |
| NLxBB | 60 | 15 | 150 | Pliable, Very good cohesion |

Table 2 shows that controlling the product hardness / pliability in hashish products made with an extruder is an interplay between operating temperature, extruder screw speed and pressure. Table 2 also shows that at a pressure of 150 PSI (which is approximately in the middle of the pressure range of Example 1), a lower extruder screw speed of 15 rpm and an operating temperature of 60° C., an extruded hashish product is obtained with good cohesion and a pliable texture.

### Comparative Example 4 - Density, Cross Section Dimensions and Weight of Pressed Hashish Units

In this example, hashish products were manufactured by pressing isolated trichomes from NLxBBHigh cannabis strain according to the pressing procedure set forth in PCT/CA2020/051733. Hashish products were tested as per the Density Test with the Sartorius Density Determination Kit as described above. The results are presented in the following tables 3A and 3B.

TABLE 3A

| Product # | Density (g/mm³) | H x W x L, (mm) | mass (g) | Unit volume (mm³) | THC Content (wt.%) |
|---|---|---|---|---|---|
| 1 | 0.91 | 5.23 x 20.94 x 20.28 | 1.98 | 2220.99 | 25.11 |
| 2 | 0.87 | 5.50x 16.08 x 23.80 | 1.85 | 2104.87 | 24.01 |
| 3 | 0.85 | 5.67 x 17.85 x 24.44 | 2.14 | 2473.56 | 23.63 |
| 4 | 0.89 | 6.08 x 18.41 x 21.64 | 2.01 | 2422.23 | 24.18 |
| 5 | 0.85 | 6.80 x 19.56 x 20.69 | 2.18 | 2751.94 | 24.57 |
| 6 | 0.85 | 5.73 x 24.14 x 19.13 | 2.12 | 2646.10 | 25.11 |
| 7 | 0.93 | 5.37 x 18.82 x 23.25 | 2.09 | 2349.72 | 24.27 |
| 8 | 0.86 | 6.34 x 19.95 x 20.49 | 2.08 | 2591.64 | 24.95 |
| 9 | 0.86 | 6.19 x 20.45 x 19.35 | 2.07 | 2449.43 | 25.47 |
| 10 | 0.92 | 5.90 x 18.46 x 22.78 | 2.16 | 2481.06 | 25.14 |
| 11 | 0.91 | 5.33 x 19.83x 21.41 | 2.04 | 2262.91 | 24.75 |
| 12 | 0.92 | 5.22 x 20.98 x 21.95 | 2.09 | 2403.87 | 25.33 |

TABLE 3B

| | Density (g/mm³) | H (mm) | W (mm) | L (mm) | Unit Volume (mm³) | Mass (g) | THC (wt.%) |
|---|---|---|---|---|---|---|---|
| Standard deviation | 0.03 | 0.50 | 2.00 | 1.71 | 182.37 | 0.09 | 0.59 |
| Average | 0.89 | 5.78 | 19.62 | 21.60 | 2429.86 | 2.07 | 24.73 |
| Coefficient of variance | 0.04 | 0.09 | 0.10 | 0.08 | 0.08 | 0.04 | 0.02 |

It was observed by the present inventor that the pressed hash floats in water. This floating property could be explained with the density of the pressed hashish products being lower than that of water, which about 1.0 g/mm³. The results show that pressing isolated trichomes results in hashish products units having some variations in respective density, weight and respective cross section dimensions compared to the average density, weight and cross section dimensions of the set of units.

### Example 4 - Density, Cross Section Dimensions and Weight of Extruded Hashish Units

In this example, hashish products were manufactured with isolated trichomes from NLxBB cannabis strain in accordance with an embodiment procedure of the present disclosure.

Extrusion was done with a 150 g kief batch with a barrel temperature of 60° C. and a rotor speed of 15 rpm with a 20 mm x 5 mm die. Hashish products were tested as per the Density Test with the Sartorius Density Determination Kit as described above. The results are presented in the following tables 4A and 4B.

TABLE 4A

| Product # | Density (g/mm³) | H x W x L, (mm) | mass in air (g) | Unit volume (mm³) | THC Content (wt.%) |
|---|---|---|---|---|---|
| 1 | 1.06 | 6.08 x 21.1 x 21.65 | 2.67 | 2777.44 | 28.10 |
| 2 | 1.06 | 6.02 x 21.1 x 21.57 | 2.67 | 2739.86 | 27.48 |
| 3 | 1.06 | 6.12 x 21.06 x 20.88 | 2.58 | 2691.16 | 27.49 |
| 4 | 1.05 | 6.14 x 21.22 x 22.36 | 2.73 | 2913.30 | 28.12 |
| 5 | 1.04 | 6.06 x 21.11 x 20.99 | 2.58 | 2685.18 | 28.41 |
| 6 | 1.05 | 6.15 x 21.32 x 20.45 | 2.54 | 2681.36 | 28.35 |
| 7 | 1.06 | 6.18 x 21.27 x 21.96 | 2.73 | 2886.61 | 27.66 |
| 8 | 1.04 | 6.11 x 21.18 x 21.15 | 2.61 | 2737.02 | 27.68 |
| 9 | 1.04 | 6.16 x 21.23 x 20.42 | 2.57 | 2670.46 | 28.35 |
| 10 | 1.05 | 6.12 x 21.01 x 21.07 | 2.60 | 2709.21 | 27.57 |
| 11 | 1.05 | 6.18 x 21.18 x 20.67 | 2.57 | 2705.55 | 27.98 |
| 12 | 1.05 | 6.17 x 21.26 x 21.48 | 2.65 | 2817.62 | 28.16 |

TABLE 4B

| | Density (g/mm³) | H (mm) | W (mm) | L (mm) | Mass (g) | Volume (mm³) | THC (wt.%) |
|---|---|---|---|---|---|---|---|
| Standard deviation | 0.01 | 0.05 | 0.09 | 0.60 | 0.06 | 81.60 | 0.32 |
| Average | 1.05 | 6.12 | 21.17 | 21.22 | 2.62 | 2751.23 | 27.96 |
| Coefficient of variance | 0.01 | 0.01 | 0.00 | 0.03 | 0.02 | 0.03 | 0.01 |

It was observed by the present inventor that the extruded hash sinks in water. This sinking property could be explained with the density of the extruded hashish products being higher than that of water. The results show that extruding isolated trichomes results in hashish products units having substantially identical density, weight and respective cross section dimensions compared to the average density, weight and cross section dimensions of the set of units.

### Comparative Example 5 - Density Variations in Peripheral and Core Samples of Pressed Hashish unit

In this example, a 5 inch X 6 inch hashish product unit was manufactured as per the pressing procedure as in comparative Example 4. Peripheral samples of about 10 mm X about 7 mm were cut from the edges of the hashish product to determine density variations across the edge of the pressed hashish product. Results are shown in the following table 5.

TABLE 5

| Sample # | Density of peripheral samples (g/mm³) | Density of core samples (g/mm³) |
|---|---|---|
| 1 | 0.96 | 0.82 |
| 2 | 0.88 | 0.82 |
| 3 | 0.82 | 0.82 |
| 4 | 0.83 | 0.86 |
| 5 | 0.83 | 0.84 |
| 6 | 0.75 | 0.82 |
| 7 | 0.79 | 0.81 |
| 8 | 0.92 | 0.86 |
| 9 | 0.87 | 0.85 |
| 10 | 0.86 | 0.83 |
| 11 | 0.80 | 0.82 |
| 12 | 0.70 | 0.83 |

The average density of peripheral samples is 0.84 g/mm³ with a SD = 0.07 and with a coefficient of variance of 8.34%. This also shows that pressing isolated trichomes results in hashish products having some variations in density values within peripheral samples of a single product unit. The average density of core samples is 0.83 g/cm³ with a SD = 0.02 and with a coefficient of variance of 1.85%. This also shows that pressing isolated trichomes results in hashish products having some variations in density values within core samples of a single product unit.

### Example 5 - Density Variations in Peripheral and Core Samples of Extruded Hashish

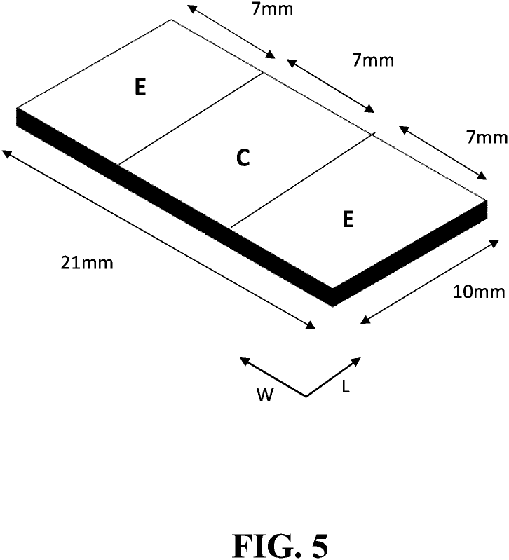

In this example, a hashish product was manufactured as in Example 4. Samples were cut from a hashish product unit having a total width of 21 mm and a length of 10 mm. Peripheral cuts were taken to make edge samples having a width of 7 mm (as shown in FIG. 5 by letter E) to determine density variations across the edge of the hashish product. Results are shown in the following table 6.

TABLE 6

| Table 6Sample# | Density of peripheral samples (g/mm³) | Density of core samples (g/mm³) |
|---|---|---|
| 1 | 1.04 | 1.02 |
| 2 | 1.03 | 1.03 |
| 3 | 1.04 | 1.05 |
| 4 | 1.03 | 1.05 |
| 5 | 1.05 | 1.03 |
| 6 | 1.05 | 1.03 |
| 7 | 1.05 | 1.02 |
| 8 | 1.05 | 1.05 |
| 9 | 1.05 | 1.02 |
| 10 | 1.05 | 1.02 |
| 11 | 1.04 | 1.03 |
| 12 | 1.04 | 1.02 |

The average density of peripheral samples is 1.04 g/mm³ with a SD = 0.01 and with a coefficient of variance of 0.01%. This also shows that extruding isolated trichomes results in hashish products having substantially identical density values within peripheral samples of a single product unit. The average density of core samples is 1.03 g/cm³ with a SD = 0.01 and with a coefficient of variance of 0.01%. This also shows that extruding isolated trichomes results in hashish products having substantially identical density values within core samples of a single product unit. Surprisingly, this also shows that core and peripheral density values are substantially identical within a single unit, which further demonstrates homogeneity of the hashish product.

### Comparative Example 6 - Dimensions Variations of 2 g Pressed Hashish Units

In this example, hashish products were manufactured as per the pressing procedure as in comparative Example 4. The hashish products were trimmed and cut to obtain 2.00 +/- 0.01 g pieces of hashish samples. Physical characterization was performed on the different hashish sample products and the results reported in Table 7.

TABLE 7

| Sample Number | Length (mm) | Width (mm) | Depth (mm) | Volume (mm3) | Mass (g) |
|---|---|---|---|---|---|
| 1 | 21 | 13 | 6 | 1638 | 2.00 ± 0.01 |
| 2 | 22 | 7 | 9 | 1386 | 2.00 ± 0.01 |
| 3 | 20 | 9 | 6 | 1080 | 2.00 ± 0.01 |
| 4 | 23 | 12 | 6 | 1656 | 2.00 ± 0.01 |
| 5 | 19 | 8 | 8 | 1216 | 2.00 ± 0.01 |
| 6 | 20 | 11 | 5 | 1100 | 2.00 ± 0.01 |
| 7 | 20 | 15 | 7 | 2100 | 2.00 ± 0.01 |
| 8 | 21 | 12 | 6 | 1512 | 2.00 ± 0.01 |
| 9 | 15 | 16 | 7 | 1680 | 2.00 ± 0.01 |
| 10 | 22 | 11 | 8 | 1936 | 2.00 ± 0.01 |
| 11 | 22 | 10 | 6 | 1320 | 2.00 ± 0.01 |
| 12 | 18 | 12 | 8 | 1728 | 2.00 ± 0.01 |
| 13 | 23 | 10 | 7 | 1610 | 2.00 ± 0.01 |
| 14 | 20 | 10 | 8 | 1600 | 2.00 ± 0.01 |

These results show that pressed hashish products made to meet a specific weight suffered from significant variations in terms of physical characteristics, such as density and / or dimensions.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1-71. (canceled)

72. A process of making a hashish product, comprising
a) providing isolated cannabis trichomes;
b) mixing the isolated cannabis trichomes in an extrusion process under conditions sufficient to obtain a resinous mixture; and
c) retrieving at least a portion of the resinous mixture through an extrusion die to obtain a hashish product comprising a substantially homogeneous cohesive mass of the isolated cannabis trichomes.

73. The process according to claim 72, wherein the isolated cannabis trichomes are kief.

74. The process according to claim 72, further comprising cutting the hashish product according to a pre-established cutting operational parameter.

75. The process according to claim 72, further comprising incorporating one or more additional components prior to, simultaneously with, or following the mixing step, wherein the one or more additional components include one or more cannabinoids, one or more terpenes, one or more flavonoids, water, one or more flavoring agents, one or more non-toxic coloring agents, or a mixture thereof.

76. The process according to claim 75, wherein the one or more cannabinoids is in the form of a crude cannabis extract, a cannabis distillate, a winterized cannabis plant extract, cannabis rosin, cannabis resin, cannabis wax, cannabis shatter, or any combination thereof.

77. The process according to claim 75, wherein the one or more cannabinoids includes a plurality of cannabinoids.

78. The process according to claim 75, wherein the one or more cannabinoids includes tetrahydrocannabinol (THC), cannabidiol (CBD), and cannabinol (CBN).

79. The process according to claim 75, wherein the water is incorporated in the form of steam.

80. The process according to claim 75, wherein the water is incorporated in the form of liquid.

81. The process according to claim 72, wherein said conditions include a selected shear.

82. The process according to claim 72, wherein said conditions further include a selected pressure selected between about 5 psi and about 500 psi.

83. The process according to claim 81, wherein said conditions further include mixing at a selected temperature of about 140° C. or less.

84. The process according to claim 72, wherein the hashish product comprises a cannabinoid content of from about 5 wt.% to about 90 wt.%.

85. The process according to claim 84, wherein the cannabinoid content is up to about 60 wt.%.

86. The process according to claim 72, wherein the isolated cannabis trichomes are from a plurality of cannabis strains.

87. The process according to claim 72, wherein the mixing includes applying compression and shear forces to the isolated trichomes via a plurality of interpenetrate helicoidal surfaces within an elongated enclosure.

88. The process according to claim 87, wherein the interpenetrate helicoidal surfaces are on at least one screw, the method further comprising adjusting a rotational speed of the at least one screw within the elongated enclosure to obtain the cohesive mass.

89. The process according to claim 88, wherein the rotational speed of the at least one screw is between about 10 rpm and about 1000 rpm.

90. A hashish product comprising a substantially homogeneous cohesive mass of isolated cannabis trichomes made by the process according to claim 72.

91. A hashish product comprising an extruded mass of trichomes made by the process according to claim 72.

92. A set of hashish products, wherein:
each hashish product comprises an extruded mass of isolated cannabis trichomes; and
a physical characteristic of each of at least 85 percent of the hashish products imparted during extrusion of the extruded mass of isolated cannabis trichomes is between 0.9 and 1.1 times an average of the physical characteristic of the hashish products.

93. The set of hashish product of claim 92, wherein the physical characteristic is:
a) a respective cross section dimension;
b) a respective unit weight;
c) a respective density measured at 20° C.

94. The set of hashish products of claim 92, wherein the physical characteristic of each of at least 90 percent of the hashish products in the set of hashish products is between about 0.9 and about 1.1 times the average of the physical characteristic in the set of hashish products.

95. The set of hashish products of claim 92, wherein the physical characteristic is between about 0.95 to about 1.05 times the average physical characteristic in the set of hashish products.

96. The set of hashish products of claim 92, wherein each hashish product in the set of hashish products comprises a cannabinoid content of between about 5 wt.% to about 90 wt.%.

97. The set of hashish products of claim 96, wherein the cannabinoid content comprises two or more of tetrahydrocannabinol (THC), cannabidiol (CBD), cannabigerol (CBG), and cannabinol (CBN).

98. The set of hashish products of claim 92, wherein the isolated cannabis trichomes are from a plurality of strains of cannabis plant.

99. The set of hashish products of claim 92, wherein the hashish products further comprise one or more additional components comprising one or more cannabinoids, one or more terpenes, one or more flavonoids, water, one or more flavoring agents, one or more non-toxic coloring agents, or any combinations thereof.

100. The set of hashish products of claim 99, wherein the one or more additional components is substantially homogenously distributed throughout the cohesive mass of at least 85 percent of the hashish products.

101. The set of hashish products of claim 99, wherein the one or more additional components is distributed on an external surface of the hashish products.

102. The set of hashish products of claim 92, wherein the set of hashish products comprises at least 10 hashish products.

103. A hashish product comprising an extruded mass of isolated cannabis trichomes, wherein a density of the hashish product is substantially homogeneous throughout the extruded mass of isolated cannabis trichomes.

104. The hashish product of claim 103, wherein each of at least 90 percent of portions in a plurality of portions of the hashish product have a respective density which is between about 0.9 to about 1.1 times an average density of the plurality of portions.

105. The hashish product of claim 104, wherein the respective density is between about 0.95 to about 1.05 times the average density of the plurality of portions.

106. The hashish product of any claim 104, wherein the respective density is measured according to the following formula:
$$d e n s i t y = \frac{W a ∗ d w}{W a - W w}$$
wherein
Wa = weight of the hashish portion in air,
dw = density of water at 20° C.,
Ww = weight of the hashish portion in water at 21° C.
